(19) United States
(12) Patent Application Publication
(10) Pub. No.: US 2011/0165128 A1
(43) Pub. Date: Jul. 7, 2011

(54) HOMING IN MESENCHYMAL STEM CELLS

(75) Inventors: Sergey V. Doronin, Stony Brook, NY (US); Irina A. Potapova, Stony Brook, NY (US); Ira S. Cohen, Stony Brook, NY (US); Michael R. Rosen, New York, NY (US); Richard B. Robinson, Cresskill, NJ (US); Peter R. Brink, Setauket, NY (US)

(73) Assignee: Columbia University in the City of New York, New York City, NY (US)

(21) Appl. No.: 12/921,396

(22) PCT Filed: Mar. 6, 2009
(86) PCT No.: PCT/US09/36414

§ 371 Date: Mar. 21, 2011

Related U.S. Application Data
(60) Provisional application No. 61/068,568, filed on Mar. 7, 2008.

(51) Int. Cl. A61K 35/12; C12N 5/0775; A61P 9/00; A61P 9/10

(52) U.S. Cl. 424/93.7; 435/325; 435/404

(57) ABSTRACT

The present invention relates to expression of CXCR4 in mesenchymal stem cells (MSCs) and homing of MSCs to sites of injury. In particular, the invention provides expanded cultures of MSCs which maintain cell surface expression of CXCR4. The MSCs are capable of homing to sites of injury and are suitable for treatment of ischemic disorders, including cardiac disorders, bone and cartilage disorders, liver disorders, inflammatory disorders, and stroke.

### CROSS REFERENCE TO RELATED APPLICATIONS

This present application is a national phase application of PCT Application No. PCT/US2009/036414, filed on Mar. 6, 2009 and claims priority of U.S. Provisional Application. 61/068,568, filed Mar. 7, 2008, the entire contents of which are hereby incorporated by reference as if fully set forth herein, under 35 U.S.C. §119(e).

### FEDERAL FUNDING

This invention was made with government support under grants HL67101 and HL28958 from the National Institutes of Health. The government has certain rights in the invention.

### FIELD OF THE INVENTION

The present invention relates to expression of CXCR4 in mesenchymal stem cells (MSCs) and homing of MSCs to sites of injury. In particular, the invention provides expanded cultures of MSCs which maintain cell surface expression of CXCR4. The MSCs are capable of homing to sites of injury and are suitable for treatment of ischemic disorders, including cardiac disorders, bone and cartilage disorders, liver disorders, inflammatory disorders, and stroke.

### BACKGROUND OF THE INVENTION

Heart failure is a notoriously progressive disease, despite medical management. The increasing gap between the incidence of end-stage heart failure and surgical treatment is due, in great part, to the shortage of donor organs. Thus, there is a need for alternative approaches for treatment of damaged heart tissue that is not dependent of the availability of donor organs.

Mesenchymal stem cells isolated from bone marrow are increasingly used as a therapeutic tool for cell or tissue replacement, including to assist in hematopoietic stem cell engraftment in the bone marrow, to treat bone and cartilage disorders, liver disorders, inflammatory disorders such as inflammatory bowel disease and Chron's disease, to treat radiation damaged tissue, and to improve functions of ischemic tissues such as after stroke or myocardial infarction.

Due to the low abundance of MSCs from bone marrow isolates, culture expanded human MSCs (hMSCs) are increasingly used in a variety of preclinical and clinical studies. However, homing capabilities of MSCs are greatly influenced by cell culture conditions and the targeting capabilities of culture expanded MSCs are limited, requiring large numbers of cells to achieve therapeutic benefits. Further, while most of freshly isolated MSCs can be detected in the bone marrow of sublethally irradiated mice after systemic administration, homing to the bone marrow of MSCs sub-cultured for as little as a 24 hours is significantly reduced, and the majority of the such systemically administered MSCs are found primarily in liver and other organs. Culturing of hMSCs for more than two passages is associated with a decrease in expression of adhesion molecules, the loss of chemokine receptors, including CXCR4, and lack of chemotactic response to chemokines. The loss of the chemokine response results in impairment of homing and represents a substantial challenge for therapeutic application of hMSCs. Accordingly, it is desired to improve targeting abilities of cultured MSCs.

### SUMMARY OF THE INVENTION

Several laboratories have shown that following isolation from bone marrow, mesenchymal stem cells quickly lose the expression of CXCR4, a receptor vital for stem cell homing. The present invention is based on the discovery that adhesion of MSCs can be stimulated by culture of the cells in conditions that maintain CXCR4 expression. Accordingly, the present invention provides novel compositions of cultured MSCs that continue to express CXCR4. The novel compositions have improved capability for engraftment in injured or ischemic tissue and may be used for treatment of disorders, including cardiac disorders.

The invention provides an expanded culture of mesenchymal stem cells in which cell surface expression of CXCR4 is maintained or induced in a substantial proportion of the cells. The expanded culture is obtained by culturing MSCs under three dimensional culture conditions such as hanging drops, such that 3D structure formation, such as spheroid formation, is induced. In an embodiment of the invention, at least about 20% of the MSCs in culture express cell surface CXCR4. In another embodiment, at least about 30% of the MSCs in culture express cell surface CXCR4. In yet another embodiment, at least about 35% of the MSCs in culture express cell surface CXCR4. Accordingly, a substantial proportion of the cells adhere to endothelial cell exposed to hypoxia.

The MSCs cultured under three dimensional culture conditions are established from MSC monolayers or from bone marrow MSCs.

The serum levels of the cultures can be adjusted to allow for expression of cytokines and other paracrine molecules, or to eliminate media components from biological sources. According to the invention, MSCs can be cultured with serum amounts ranging from serum free up to about 20% fetal bovine serum (FBS). In one embodiment, the culture comprises about 5% fetal bovine serum (FBS). In another embodiment, the amount of FBS is about 5% or less. In yet another embodiment, the culture is serum free.

Significant CXCR4 expression is observed after about 24 hours of culture. In particular embodiments of the invention, the spheroids are collected and dissociated after about 2 days of hanging drop culture, or after about 3 days of hanging drop culture, or after less than about 4 days of hanging drop culture.

In order to minimize loss of CXCR4 expression and homing ability, once harvested, the MSCs are promptly administered to a subject or frozen and stored.

For administration to a subject, the invention provides a pharmaceutical composition comprising a population of mesenchymal stem cells, as described above, in which a substantial proportion of the cells express cell surface CXCR4, and a pharmaceutically acceptable carrier.

In certain embodiments of the invention, the pharmaceutical composition is provided in an amount effective for cell or tissue replacement, or to assist in hematopoietic stem cell engraftment in the bone marrow, or to treat bone and cartilage disorders, or to treat liver disorders, or to treat inflammatory disorders such as inflammatory bowel disease and Chron's disease, or to treat radiation damaged tissue, or to improve functions of ischemic tissues such as after stroke or myocardial infarction.

The invention provides a method of preparing an expanded culture of mesenchymal stem cells that substantially express CXCR4 and are capable of adhering to epithelial cells exposed to hypoxia. The culture is prepared by obtaining proliferating mesenchymal stem cells and culturing the mesenchymal stem cells in three dimensional culture for a sufficient time that a substantial proportion of the mesenchymal cells express cell surface CXCR4. In one embodiment, the initial population of mesenchymal stem cells is from a monolayer. In another embodiment, the initial population of mesenchymal stem cells is from bone marrow. The mesenchymal stem cells are cultured in hanging drops to induce or maintain cell surface CXCR4 expression, collected, optionally dissociated, and promptly used or frozen for storage. In certain embodiments, the cells are cultured in hanging drops for about 2 days, or about 3 days, or less than about 4 days.

According to the invention, to be used in treatment of a subject, the cell surface CXCR4-expressing cells are usually dissociated from spheroids at the end of three dimensional culture, and are promptly administered to a subject or frozen for storage at the end of three dimensional culture.

The expanded culture is suitable for cell or tissue replacement, to assist in hematopoietic stem cell engraftment in the bone marrow, to treat bone and cartilage disorders, liver disorders, inflammatory disorders such as inflammatory bowel disease and Chron's disease, radiation damaged tissue, or to improve functions of ischemic or hypoxic tissues such as after stroke or myocardial infarction.

The invention further provides a method of preferentially targeting MSCs to hypoxic tissue in a subject by culturing the MSCs under three-dimensional culture conditions for a time sufficient to induce cell surface expression of CXCR4 in a substantial proportion of the cells, and administering a therapeutically effective amount of the cultured MSCs to the subject. The CXCR4 expressing cells are thus preferentially targeted to the hypoxic tissue. In an embodiment of the invention, the subject is a human.

The invention also provides a method of for cell or tissue replacement, or to assist in hematopoietic stem cell engraftment in the bone marrow, or to treat bone and cartilage disorders, or to treat inflammatory disorders such as inflammatory bowel disease and Chron's disease, or to treat radiation damaged tissue, or to improve functions of ischemic or hypoxic tissues such as after stroke or myocardial infarction, by administering an effective amount of an MSC composition of the invention. In one embodiment, the subject is a human.

The invention also provides a kit comprising mesenchymal stem cells, in which a substantial proportion of the cells express cell surface CXCR4, a physiologically acceptable carrier, and directions for administering the cells to a subject. In one embodiment of the kit, the cells are aggregated a spheroids, and are optionally dissociated upon administration. In another embodiment of the kit, the mesenchymal stem cells are dissociated.

### DESCRIPTION OF THE FIGURES

FIG. 1 depicts side and forward light scatter analysis of hMSCs from a monolayer, and from one day old (SPH1), two day old (SPH2), and three day old (SPH3) spheroids.

FIG. 2 shows the effects of trypsinization on the expression of cell surface markers by hMSCs. Cells were grown in monolayer, treated with trypsin-EDTA for 5, 30, and 90 min labeled with FITC- or PE-conjugated monoclonal antibodies specific for proteins indicated in each histogram and analyzed by flow cytometry. Grey filled histograms correspond to hMSCs trypsinized for 5 min. Thin and thick black line histograms represent hMSCs that were trypsinized for 30 and 90 minutes, respectively. Solid black histograms represent isotype controls.

FIG. 3 shows a comparison of cell surface marker expression by hMSCs and three-day old hMSC spheroid cells. Cells were isolated by trypsinization for 90 min., labeled with FITC- or PE-conjugated monoclonal antibodies and analyzed by flow cytometry. Black and grey filled histograms represent cells from a monolayer of hMSCs and the spheroids, respectively.

FIG. 4 shows expression of CD34, CD184, CD49b, and CD49d by hMSCs from a monolayer and one-, two-, and three-day old hMSC spheroids. Cells were dissociated from a monolayer (hMSCs), one day old hMSC spheroids (SPH1), two day old hMSC spheroids (SPH2), and three day old hMSC spheroids (SPH3), and stained with CD34-FITC-CD184-PE and CD49b-FITC-CD49d-PE pairs of antibodies. Representative two-color fluorescence dot plots and corresponding isotype controls are shown.

FIG. 5 depicts expression of CD184, CD34, CD49d and CD49b by cells from a monolayer of hMSCs and hMSC spheroids. Cells were dissociated using trypsin-EDTA for 90 min, stained with IgG2a-PE-IgG1-FITC; CD184-PE-CD34-FITC; CD49d-PE-CD49b-FITC antibody pairs and analyzed by flow cytometry. Representative two-color fluorescence dot plots are shown. Control stainings with isotype- and fluorochrome-matching antibody pairs are shown in panels A and D for hMSCs from a monolayer and 3 day old spheroids (SPH3), accordingly. Panels B and C show dot plots for a monolayer of hMSCs stained with CD49d-PE-CD49b-FITC (panel B) and CD184-PE-CD34-FITC (panel C) pairs. Corresponding stainings for cells from SPH3 are shown in panels E and F. Panel G shows a mean value ±SD of a percentage of CD49b positive cells from a monolayer of hMSCs and SPH3 as an average of three independent experiments. Panel H shows a mean value ±SD of a percentage of CD49d positive cells from a monolayer hMSCs and SPH3 as an average of three independent experiments. Panel I shows a mean value ±SD of a percentage of CD184 positive cells from a monolayer of hMSCs and SPH3 as an average of three independent experiments. To calculate a percentage of positively stained cells data were gaited as shown. Statistically significant changes are denoted by asterisks (*, t-test, p-value <0.05).

FIG. 6 depicts secretion of SDF-1 by a monolayer of hMSCs and hMSC spheroids. Concentrations of SDF-1 were measured in media conditioned by a monolayer of hMSCs and one (SPH1), two (SPH2) and three (SPH3) day old hMSC spheroids using ELISA and normalized to the total number of cells. Mean values of relative secretion ±SD of SDF-1 by hMSCs (n=4), SHP1 (n=8), SPH2 (n=4) and SPH3 (n=4) are shown. Statistically significant changes in comparison with hMSCs are denoted by asterisks (*, t-test, p-value <0.05).

FIG. 7 shows changes in CD184, CD49d and CD49b expression by cells from the spheroids after plating them as a monolayer. Cells were dissociated from 3 day old hMSC spheroids (SPH3) and cultured in a monolayer for 1-6 days (SPH3+1-6). Mean values of a percentage ±SD of CD184, CD49d, CD49b positive cells were determined as shown in FIG. 1. Panel A shows changes in the expression of CD184. Panel B shows changes in the expression of CD49b and CD49d. Corresponding values for a monolayer of hMSCs (hMSCs) and isotype controls are presented on each panel. Data represent an average of at least three independent experiments. Statistically significant changes in comparison with hMSCs are denoted by asterisks (*, t-test, p-value<0.05).

FIG. 8 shows intracellular localization of CXCR4. Cells were dissociated by trypsinization, plated on Lab-Tek II chamber CC2 glass slides and allowed to attach for 8 hours. After serum starvation, cells were treated with and without 1 μg/ml SDF-1α 0 for 45 min. hMSCs from a monolayer (hMSCs) or 3 day old hMSC spheroids (SPH3) were stained with anti-human CXCR4 antibody and Alexa Fluor 488-conjugated F(ab′)2 secondary antibody (green). F-actin was stained with Alexa Fluor 594-conjugated phalloidin (red). The nuclei were counterstained with DAPI (blue). Panel A shows CXCR4 staining (green) in hMSCs and SPH3. Panel B shows CXCR4 (green) and F-actin (red) staining in SPH3 cells before (untreated) and after treatment with 1 μg/ml SDF-1α for 45 min (SDF-1). Panel C shows staining for CXCR4 (green) and F-actin (red) in lamellipodia of cells from SPH3 treated with SDF-1. Sites of co-localization are denoted by white arrows.

FIG. 9 demonstrates the effects of SDF-1 on activation of ERK-1,2. Cells from a monolayer of hMSCs (hMSCs) or 3 day old hMSC spheroids (SPH3) were dissociated, plated and allowed to adhere for 8 hours. After serum starvation, cells were treated with 1 μg/m1 SDF-1α □ for 0-20 min and lysed. Proteins were separated on a SDSPAG gel and analyzed by Western blot using antibodies against total ERK-1,2 and pERK-1,2. Panel A shows pERK-1,2 and total ERK-1,2 staining for cells isolated from SPH3. Panel B shows pERK-1,2 and total ERK-1,2 for hMSCs. Panel C shows the results of densitometric analysis of pERK-1,2 for hMSCs and SPH3 after normalization for total ERK-1,2 in corresponding cellular lysates.

FIG. 10 shows adhesion of hMSCs to HUVECs. hMSCs from a monolayer (hMSCs) or from 3 day old hMSC spheroids (SPH3) were dissociated by trypsinization, labeled with Calcein AM and incubated with a monolayer of HUVECs. Prior to analysis HUVECs were exposed to normoxia or hypoxia. Adhesion of hMSCs or SPH3 cells was studied with or without 0.5 μg/ml SDF-1α □ or □ 10 □μM AMD-3100. Panel A shows effect of HUVEC pre-exposure to hypoxia on the adhesion of hMSCs (n=30). Panel B shows effects of SDF-1α and AMD3100 on the adhesion of hMSCs to normoxic HUVECs (n=30). Panel C shows effects of SDF-1α and AMD-3100 on the adhesion of hMSCs to HUVECs pre-exposed to hypoxia (n=30). There was no statistically significant difference in hMSC adhesion to HUVECs in any of the conditions described above.

### DETAILED DESCRIPTION

The present invention provides methods and compositions relating to culture expanded MSCs that maintain expression or are induced to express proteins responsible for cell adhesion and motility. While MSCs are abundant in bone marrow, the number that can be obtained from bone marrow is insufficient for clinical uses. MSCs from bone marrow can be placed in cell culture in order to increase their number, but while they retain certain stem cell characteristics and the ability to differentiate into certain cell types, expression of certain cell surface markers, such a CXCR4, is lost, concomitant with loss of homing ability, and the ability to differentiate into certain cell types. It has been demonstrated that MSCs lose CXCR4 expression shortly after isolation and only a fraction of cultured MSCs are CXCR4 positive. According to the present invention, cell culture conditions can be chosen which prevent or reverse the loss of CXCR4 expression in culture expanded MSCs. Unlike MSCs cultured under conditions that do not maintain CXCR4 expression, the cultured MSCs provided by the invention adhere preferentially to endothelial cells pre-exposed to hypoxia, and are stimulated to adhere to normoxic endothelial cells by CXCR4 agonists such as SDF-1. The CXCR4-expressing cultures are collected at a time when CXCR4 expression is high and promptly administered or packaged and frozen for storage to avoid a decrease in CXCR4 expression and homing capacity that would otherwise occur over time.

Human MSCs (hMSCs) to be used in the practice of the invention can be allogeneic and may be purchased from any reputable supplier such as Cambrex BioScience (East Rutherford, N.J.) or Clonetics/Bio Whittaker (Walkersville, Md.). Alternatively, hMSCs may be derived from bone marrow aspirates from the subject or from a healthy volunteer. For example, 10 ml of marrow aspirate is collected into a syringe containing 6000 units of heparin to prevent clotting, washed twice in phosphate buffer solution (PBS), added to 20 ml of control medium (DMEM containing 10% FBS), and then centrifuged to pellet the cells and remove the fat. The cell pellet is then resuspended in control medium and fractionated at 1100×g for 30 min on a density gradient generated by centrifugation of a 70% percoll solution at 13,000×g for 20 minutes. The mesenchymal stem cell-enriched, low density fraction is collected, rinsed with control medium and plated in Mesenchymal Stem Cell Growth Media (Cambrex Bio-Science) at 37° C. in a humidified atmosphere of 5% CO₂. Preferably, passages 2-5 of are used.

MSCs of the invention are characterized by expression of CXCR4, and are obtained by culture in three-dimensional (3D) culture. 3D culture is distinct from culture in monolayers in that it provides for aggregation of cells in a group forming a 3D space. A typical, non-limiting, 3D structure is what is referred to in the art as an “embryoid body (EB) cell aggregate” or “spheroid.” A preferred method for making hMSC spheroids of the invention is to culture hMSCs in hanging drops. In one embodiment hMSCs are cultured in 40 μl A drops, containing 250,000 cells per drop, for three days with daily changes of media.

The microenvironment of cells in 3D culture is thought to differ from that in a monolayer is several ways. For example, it is thought that the availability of oxygen and nutrients to interior cells may be reduced. Further the shape of a cell in a 3D cell mass, and the external forces acting upon it, is likely to differ from that of a cell attached to a solid support. Accordingly, other 3D culture methods can be used, provided that they promote maintenance or induction of CXCR4.

Expression of CXCR4 by MSCs is inversely correlated with expression of SDF-1. For example, in a microarray experiment to detect mRNAs that are expressed differently in monolayers and spheroids, mRNA for CXCR4 was increased 56 fold while SDF-1 mRNA expression was decreased 10 fold in cells from spheroids. As demonstrated herein, compared to hMSCs from a monolayer, a much larger proportion of hMSCs grown in hanging drop culture stain positively for CXCR4 (CD184) and the α2 integrin subunit (CD49b). Useful enriched cultures are those in which at least about 10%, at least about 20%, or at least about 30%, or at least about 35% of cells from the spheroid culture stain positive for CXCR4, a substantial increase relative to monolayer-derived cells. Measured by expression of CD49b, useful enriched cultures are those in which at least about 40%, or at least about 60%, or at least about 75%, or at least about 90% stain positive for CD49b. At the same time, the proportion of cells that express CD49d decreases. Whereas a large majority (about 71%) of cells from a monolayer stain positive to CD49b, only about 2% stain positive from 3 day old spheroids. In an embodiment of the invention, less than about 25%, or less than about 15%, or less than about 10%, or less than about 5% of the cell population expresses CD49d.

According to the invention, large numbers of MSCs that express cell surface CXCR4 and are suitable for administration to a subject are provided. MSCs are often propagated in tissue culture monolayers. However, only 5% or less of hMSCs propagated in monolayers express CXCR4, and MSCs that express little cell surface CXCR4 have impaired homing characteristics and are inefficient at targeting damaged tissue. As demonstrated herein, MSC populations which display little cell surface CXCR4,perhaps as a result of having been cultured under conditions that favor cell proliferation, can be induced to express cell surface CXCR4, and regain homing characteristics.

Accordingly, the invention provides a method for making expanded cultures of MSCs in which a substantial proportion express cell surface CXCR4. For example, as demonstrated herein, MSCs which have been proliferating in monolayers can be cultured under 3D culture conditions. After a sufficient time, usually 2-3 days, there is a significant increase in cell surface CXCR4 expression, and the homing characteristics of the MSCs are greatly improved. The MSC culture should not be maintained for an extended period of time. The MSC aggregates should be collected and used (or frozen) early enough that CXCR4 expression does not decline and cell in the aggregate do not differentiate. Typically, these undesirable characteristics begin to be observed at around the fourth day of culture.

In one embodiment, hMSCs grown as a monolayer are dissociated, collected by centrifugation, and resuspended in a suitable medium. One such medium is high-glucose Dulbecco's modified Eagle's medium (DMEM) supplemented with penicillin-streptomycin and 5% fetal bovine serum. Hanging drops are then prepared from the dissociated culture and incubated for a time sufficient to induce CXCR4 expression in a substantial proportion of cells. The MSCs regain the ability to bind to HUVEC cells cultured under anoxic conditions, and also bind, in the presence of added SDF-1, to HUVEC cultured under normoxic conditions. Other suitable media can be used, provided that is supports CXCR4 expression in a substantial proportion of MSCs. In certain embodiments, for example, in applications where it is desirable to minimize or eliminate biological products from other organisms, it is possible to limit or eliminate fetal bovine serum and still maintain production of cytokines. For example, maximum VEGF production is attained in MSC spheroids using about 5% FBS, production of cytokines is much less dependent on FBS concentration, and production of paracrine factors is sustained by factors secreted in the spheroids even at low serum concentrations. In an embodiment of the invention, the FBS concentration is about 5%. In another embodiment of the invention, the amount of FBS is less than about 5%. In another embodiment of the invention, the media is serum free.

When cultured in a monolayer, the expression of certain cytokines and paracrine factors that promote cell migration depends on serum concentration. However, in hanging drops, the production of cytokines by MSC spheroids is less dependent on serum concentration. For example, serum has no effect on angiogenin production by spheroid cultures. Also, while VEFG secretion is stimulated by the presence of serum, full stimulation is attained with 5% serum, and there is no need for higher amounts.

The present invention further provides pharmaceutical compositions comprising expanded MSCs of the invention and a pharmaceutically acceptable carrier. Pharmaceutically acceptable carriers are well known to those skilled in the art and include, but are not limited to, 0.01-0.1 M and preferably 0.05 M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Preservatives and other additives, such as, for example, antimicrobials, antioxidants and chelating agents may also be included with all the above carriers.

CXCR4-expressing MSCs can also be incorporated or embedded within scaffolds that are recipient-compatible and which degrade into products that are not harmful to the recipient. These scaffolds provide support and protection for CXCR4-expressing MSCs that are to be transplanted into the recipient subjects. Natural and/or synthetic biodegradable scaffolds are examples of such scaffolds. Accordingly, the present invention provides methods for promoting tissue repair, wherein CXCR4-expressing MSCs are incorporated within scaffolds, prior to transplantation into a subject in need of tissue repair.

A variety of different scaffolds may be used successfully in the practice of the invention. Such scaffolds are typically administered to the subject in need of treatment as a transplanted patch. Preferred scaffolds include, but are not limited to biological, degradable scaffolds. Suitable synthetic material for a cell transplantation scaffold must be biocompatible to allow migration and preclude immunological complications, and should be able to support cell growth and differentiated cell function. It may also be resorbable, allowing for a completely natural tissue replacement. The scaffold should be configurable into a variety of shapes and should have sufficient strength to prevent it from collapsing or from pressure-induced bursting upon implantation.

Uses and Administration of the Composition

The present invention provides for delivery and tracking of spheroid-derived MSCs of the invention. Labeled MSCs provide a means for tracking the distribution and fate of MSCs that have been administered to a subject to promote cardiac repair.

Mesenchymal stem cells are believed to migrate via the bloodstream to seed the sites of hematopoiesis during embryonic development or sites of injury in adult life. MSCs express a variety of adhesion molecules and respond to CXCL12 (SDF-1), CX3CL1, CXCL16, CCL3, CCL19 and CCL21 chemokines. The chemokine receptor 4 (CXCR4) and its ligand, the stromal cell-derived factor-1 (SDF-1), are believed to be key players in the process. The interaction between SDF-1 and CXCR4 is reported to mediate engraftment of cancer stem cells to the bone marrow, chemotaxis of endothelial and neuronal cells, trafficking of rat MSCs to sites of injury and the migration of MSCs in vitro.

MSCs are useful as a tool to treat bone and cartilage disorders, and when infused into peripheral circulation, are attracted to injured tissues. Systemic administration of MSCs also improves functions of ischemic tissues after stroke or myocardial infarction.

The present invention provides methods and compositions which maybe used for targeting diseased tissue and for treatment of various diseases. The compositions are used for cell or tissue replacement, including, but not limited to assisting in hematopoietic stem cell engraftment in the bone marrow, treating bone and cartilage disorders, liver disorders, inflammatory disorders such as inflammatory bowel disease and Chron's disease, treating radiation damaged tissue, and improving functions of ischemic tissues such as after stroke or myocardial infarction. According to the invention, MSCs that express CXCR4 adhere to vascular endothelial cells exposed to hypoxic conditions. Accordingly, MSC compositions of the invention which contain substantial numbers of CXCR4 positive cells, are useful for targeting hypoxic tissue in a subject.

The term “cardiac disorder” as used herein refers to diseases that result from any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathy), diseases such as angina pectoris and myocardial ischemia and infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart (for example, atrial septal defect). For further discussion, see Braunwald, Heart Disease: a Textbook of Cardiovascular Medicine, 5th edition, W B Saunders Company, Philadelphia Pa. (1997) (hereinafter Braunwald). The term “cardiomyopathy” refers to any disease or dysfunction of the myocardium (heart muscle) in which the heart is abnormally enlarged, thickened and/or stiffened. As a result, the heart muscle's ability to pump blood is usually weakened. The disease or disorder can be, for example, inflammatory, metabolic, toxic, infiltrative, fibroplastic, hematological, genetic, or unknown in origin. There are two general types of cardiomyopathies: ischemic (resulting from a lack of oxygen) and nonischemic. Other diseases include congenital heart disease which is a heart-related problem that is present since birth and often as the heart is forming even before birth or diseases that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. Specific cardiac disorders to be treated also include congestive heart failure, ventricular or atrial septal defect, congenital heart defect or ventricular aneurysm. The cardiac disorder may be pediatric in origin. The cardiac disorder may require-ventricular reconstruction.

The methods of the invention comprise administration of hMSCs that express CXCR4 in a pharmaceutically acceptable carrier, for cell or tissue replacement, and particularly for treatment of conditions associated by ischemia and hypoxic tissue, including cardiac disorders. Non-limiting examples of disorders to be treated include bone and cartilage disorders, liver disorders, inflammatory disorders, radiation exposure, bone marrow transplants, ischemic tissue after stroke, myocardial infarction, and peripheral vascular damage.

“Administering” means delivering in a manner which is effected or performed using any of the various methods and delivery systems known to those skilled in the art. Administering can be performed locally or systemically, for example, pericardially, intracardially, subepicardially, transendocardially, via implant, via catheter, intracoronarily, intravenously, intramuscularly, subcutaneously, parenterally, intraperitoneally, intrathecally, intralymphatically, intralesionally, or epidurally. Administering can also be performed, for example, once, a plurality of times, and/or over one or more extended periods.

The hMSC aggregates are collected from hanging drops, dispersed in a suitable carrier, and administered. Alternatively, the hMSCs can be collected, dissociated, and packaged in a carrier suitable for freezing and administration, and frozen for storage or shipping. For use, the cells are thawed and administered. If the freezing solution is not compatible with administration to a subject, the frozen cells are thawed and collected, for example by centrifugation, and a suitable carrier is substituted. The hMSC aggregates can also be frozen for storage, and the aggregates dissociated at the time of use.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Aqueous dextrose and glycerol solutions can also be employed as liquid carriers, particularly for injectable solutions. Examples of suitable pharmaceutical carriers are described in “Remington's Pharmaceutical Sciences” by E. W. Martin. Such compositions will contain a therapeutically effective amount of MSCs, together with a suitable amount of carrier so as to provide the form for proper administration to the patient. The formulation should suit the mode of administration.

A “therapeutically effective amount” is an amount sufficient to treat an ischemic disorder, such as a bone or cartilage disorder, a liver disorder, an inflammatory disorder, a cardiac disorder such as myocardial infarction, stroke, or vascular damage, or radiation damage. The appropriate concentration (i.e., number and volume of MSCs) of the composition of the invention which will be effective in the treatment of a particular disorder or condition will depend on the nature of the disorder or condition and the route of administration. Although migratory MSCs will usually be systemically administered, the particular location at which they are administered, and characteristics of circulation at the target, may affect the cell number or volume. Also, in some cases, it may be desirable to administer MSCs of the invention directly to a desired location, for example by injection directly into a tissue, and it might be preferred to administer fewer cells, possibly at high concentration, relative to intravenous administration. The amount of cells infused is usually determined by kg of body weight. MSCs from expanded populations of bone marrow aspirates that express little or no cell surface CXCR4 are typically administered systemically in doses of from about 1×10⁶ to about 1×10⁷ MSCs per kg of body weight. MSCs of the invention are effective at those doses, but can be administered in amounts that are significantly less as they are substantially enriched in migration competent cells that express cell surface CXCR4. Thus MSCs or the invention, can be administered in doses of from about 2×10⁴ MSCs per kg of body weight to about 1×10⁶ MSCs per kg of body weight. Preferably, the dose of MSCs is from about 5×10⁴ MSCs per kg of body weight to about 5×10⁵ per kg of body weight or from about 1×10⁵ MSCs per kg of body weight to about 3×10⁵ per kg of body weight. In one embodiment of the invention, MSCs from 40 spheroids (about 1×10⁷ MSCs), of which about 20% or more express cell surface CXCR4, are administered to a subject (about 1×10⁵ MSCs per kg of body weight).

When injected directly into a tissue, the MSCs of the invention can be used just as MSCs from bone marrow aspirates that express little or no cell surface CXCR4. Typically, those cells are used in amounts that ranges from about 10⁶ to about 5×10⁸, depending on the site of injection. For example, a range that might be used for transendocardial injection is from 10⁷ to about 2×10⁸ cells, and the dose may be divided and injected at several locations in the tissue. The MSCs of the invention, which are enriched in cell that express CXCR4, can also be used in lesser amounts, such as, for example, from about 5×10⁴ to about 2×10⁷ MSCs, or from about 10⁵ to about 10⁷ MSCs, or from about 5 10⁵ to about 5×10⁶ MSCs. The amounts and volumes can be determined by one of skill in the art using standard clinical techniques. In addition, in vitro assays may optionally be employed to help identify optimal dosage ranges. The precise dose to be employed in the formulation will also depend on the proportion of CXCR4 expressing cells in the composition, and on the route of administration, and should be decided according to the judgment of the practitioner and each patient's circumstances. Effective doses may be extrapolated from dose response curves derived from in vitro or animal model test systems. Additionally, the administration of the composition could be combined with other known efficacious drugs if the in vitro and in vivo studies indicate a synergistic or additive therapeutic effect when administered in combination.

The progress of the recipient receiving the treatment may be determined using assays that are designed to test restoration of injured or ischemic tissue. For restoration of cardiac function, such assays include, but are not limited to ejection fraction and diastolic volume (e.g., echocardiography), PET scan, CT scan, angiography, 6-minute walk test, exercise tolerance and NYHA classification.

Quantum dots (QDs) provide a convenient labeling means for MSCs QDs are semiconductor nanoparticles that were discovered in the early 1980's. QDs used for biological applications consist of a cadmium selenide or cadmium tellurium semiconductor core, a zinc sulfide inner shell and an outer polymer coating. The result is a water-soluble particle 13-15 nm in diameter. QDs provide a useful fluorescent tracking signal that can be deliver into the cytosol of an MSC through a passive endocytosis-mediated delivery system, and the labeled cells can be tracked in vivo for up to at least eight weeks. See, e.g., U.S. Application Ser. No. 60/919,593.

The invention provides a kit containing the novel MSC compostions of the invention. Typically, the kit contains frozen MSCs packaged in containers ready for administration with little or no further preparation. For example, the container can be a vial or syringe. To allow for easy substitution of carrier, for example, to replace freezing solution with a carrier suitable for administration, the MSCs may be packaged in centrifuge tubes or other container that facilitates media change. A kit comprising MSC aggregates might further contain a dissociation solution, such as 0.25% trypsin-EDTA, a tripsin inibitor solution, and a carrier solution suitable for administration. The kit also contains instructions for use.

The present invention is not to be limited in scope by the specific embodiments described herein which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Throughout this application, various publications are referenced. The disclosures of these publications in the entireties are hereby incorporated by reference into this application in order to more fully describe the state of the art to those skilled therein as of the date of the invention described and claimed herein.

### EXAMPLES

It is to be understood and expected that variations in the principles of invention herein disclosed may be made by one skilled in the art and it is intended that such modifications are to be included within the scope of the present invention. The following examples only illustrate particular ways to use the novel red fluorescent protein of the invention, and should not be construed to limit the invention.

### Example 1

Cell culture: hMSCs and HUVECs were purchased from Cambrex BioScience. HMSCs were cultured at 37° C. in a humidified atmosphere of 5% CO₂ in Mesenchymal Stem Cell Growth Media (Lonza). HUVECs were cultured in EGM-2 basal media containing growth factors supplied by the manufacturer (Lonza). Passages 2 to 5 of hMSCs or HUVECs were used.

Spheroids were formed from hMSCs as previously described (Potapova, I. S., 2007, Stem Cells 25:1761-8). Briefly, hMSCs grown to confluence were washed with Dulbecco's phosphate buffered saline (PBS) (Sigma) and dissociated with 0.25% trypsin-EDTA solution (Lonza). The digestion was stopped by addition of trypsin inhibitor solution (Lonza). hMSCs were collected by centrifugation and resuspended in high glucose Dulbecco's Modified Eagle's Medium (DMEM) (Sigma) supplemented with penicillin-streptomycin (Sigma) and 5% fetal bovine serum (Sigma). The cells (250,000 cells in 40 μl) were kept for 3 days in hanging drops. Growth media was changed every day.

Flow cytometric analysis of cell surface antigens: Analysis of cell surface antigens was performed as described (Majumdar, M. K., 2003, J. Biomed. Sci. 10:228-41) with some modifications. Cells were dissociated from a monolayer of hMSCs or hMSC spheroids with 0.25% trypsin-EDTA solution (Lonza) for 90 min at room temperature. Trypsinization was stopped by addition of trypsin inhibitor solution (Lonza). Cells were collected by centrifugation and washed with flow cytometry buffer made from PBS containing 2% bovine serum albumin (BSA) (Sigma) and 0.1% sodium azide (Fluka). Cells (2×10⁵) were stained using manufacturer suggested concentrations of fluorochrome-conjugated monoclonal antibodies for 30 min at room temperature in the dark. Anti-human HLA class I, CD31, CD34, CD55, CD105 were purchased from Diaclone. Anti-human c-met was obtained from eBioscience. Antibodies to human CD28, CD29, CD38, CD44, CD49b, CD49d, CD54, CD73, CD90, CD117, CD166, CD184, and CD209 were from BD Biosciences. After staining, cells were washed with 5 ml of the flow cytometry buffer and resuspended in the flow cytometry buffer supplemented with 1% paraformaldehyde (Electron Microscopy Sciences). Background staining was assessed by incubation of cells with mouse fluorochrome- and isotype-matched immunoglobulins (isotype controls).

Flow cytometric analysis of hMSCs grown as a monolayer or isolated from the spheroids was performed using identical instrumental settings by analyzing 10,000 events on a FACScan flow cytometer (Becton Dickinson). Signals from subcellular debris were eliminated during data acquisition by gating. The CellQuest™ software package was used to process the data.

Exposure of cells to hypoxia: HUVECs were exposed to hypoxia in a BD GasPak EZ Anaerobe Gas Generating Pouch System with an Indicator (BD Biosciences) as described (Potapova, 2007). As certified by the manufacturer, the Anaerobe Gas Generating Pouch System produces an atmosphere containing 10% carbon dioxide and 1% oxygen.

Preparation of conditioned medium: DMEM containing 5% fetal bovine serum was conditioned by a confluent monolayer of hMSCs or by hMSC spheroids for 24 h as described (Potapova, 2007). Conditioned medium was collected, passed through Acrodisc 0.2 um HT Tuffryn Membrane Low Protein Binding Filters (Pall Corporation) and stored at −80° C. until use.

SDF-1 ELISA: The SDF-1 content in conditioned medium was measured with an ELISA kit for human SDF-1 (R&D systems). The ELISA was performed according to the manufacture's protocol.

Immunocytochemistry: Cells were dissociated from a monolayer of hMSCs or 3 day old hMSC spheroids as described above. Cells were collected by centrifugation, washed with PBS, plated in DMEM containing 5% fetal bovine serum on Lab-Tek II chamber CC2 glass slides (Nalge Nunc International) and allowed to adhere for 8 hours. Then the cells were serum starved for 2 h in Hank's Balanced Salt Solution (HBSS) and treated with or without 1 μg/ml SDF-la in HBSS for 45 min in a humidified atmosphere of 5% CO₂ at 37° C. Cells were fixed with buffered formalin (1:10 dilution) (Fisher Diagnostic) for 10 min, washed with PBS and permeabilized with 0.1% Triton X-100 in PBS for 5 min at room temperature. Slides were washed three times with PBS and blocked with 5% BSA in PBS for 30 min. BSA was than removed, slides were washed again with PBS and cells were stained overnight at 4° C. with 1 μg/ml anti-human CXCR4 antibody (R&D Systems; catalog number MAB172). Unbound CXCR4 antibodies were removed by washing slides three times with PBS. Slides were then incubated with anti-mouse Alexa Fluor 488 conjugated F(ab')2 secondary antibodies (Molecular Probes, 1:1000) dissolved in PBS containing 0.165 μM Alexa Fluor 594 phalloidin (Molecular Probes) and 1% BSA for 2 hours at room temperature in the dark. Slides were washed three times with PBS and mounted on coverslips in VectaShield mounting media containing 4,6-diamidino-2-phenylindole (VectaShield-DAPI, Vector Laboratories).

Immunofluorescence was analyzed by deconvolution microscopy using an Axiovert 200 M fluorescence microscope (Carl Zeiss). Cross-sectional images were obtained with 250 nm Z-stack steps and processed using a constrained iterative algorithm of the AxioVision 4.1 software package (Carl Zeiss).

ERK-1,2 activation assay: Cells were dissociated from a monolayer of hMSCs or 3 day old hMSC spheroids as described above, collected by centrifugation and washed with PBS. Dissociated cells were plated in 24 well tissue culture dishes (5×10⁴ per well) in DMEM containing 5% fetal bovine serum and allowed to adhere for 8 hours. Cells were then serum starved for 4 hours and treated with and without 1 μg/ml SDF-1α in HBSS for 0-20 min at 37° C. in a humidified atmosphere of 5% CO₂. Cells were lysed with 1 ml per well of lysis buffer containing 0.025 M Tris-HCl, pH 7.4, 0.15 M NaCl, 5 mM EDTA, 1% Triton X-100, 0.5% NP-40 and a set of protease inhibitors (Roche) and phosphatase inhibitors (cocktails type 1 and 2) (Sigma) for 15 min at 4° C. The extract was cleared by centrifugation at 15,000 g at 4oC for 30 min. Proteins (25 μg) were separated in Bis-Tris 12% Criterion gel using XT MOPS running buffer (Bio-Rad) and transferred onto nitrocellulose membranes (Bio-Rad). Western blot was performed using p44/42 MAP kinase and phospho-p44/42 MAP kinase (T202/Y284) (197G2) antibodies (Cell Signaling Technology) Immunoreactive bands were visualized using affinity purified peroxidase labeled goat anti-rabbit F(ab')₂ fragment antibody (Kirkegaard&Perry Laboratories) and ECL Western Blotting Detection Reagents (GE Healthcare).

Adhesion Assay: Adhesion of hMSCs to endothelial cells was studied using hMSCs isolated from a monolayer or 3 day old hMSC spheroids. Cells were dissociated using 0.25% trypsin-EDTA solution (Lonza) for 15 min in the case of hMSC monolayers, or for 90 min in the case of the spheroids. Trypsinization was stopped by addition of trypsin inhibitor solution (Lonza). Isolated cells were washed with PBS, labeled with 2 μg/ml Calcein AM in HBSS for 45 min at 37oC in a humidified atmosphere of 5% CO2, washed with HBSS and resuspended in DMEM supplemented with 5% fetal bovine serum. HUVECs were plated in 24 well plates and maintained in EBM-2 growth media for 2-3 days until they reached confluence. For the adhesion assay, EBM-2 media was replaced with 0.5 ml of DMEM supplemented with 5% fetal bovine serum. hMSCs (10,000 cells per well) were added to endothelial cells. The adhesion assay was performed in the presence 0.5 μg/ml SDF-1, 10 μM AMD3100 or without additives. Plates were placed on a rotation platform (30 rotations per min) and incubated for 30 min at 37° C. Wells were washed three times with 1 ml of DMEM supplemented with 5% fetal bovine serum followed by addition of 0.5 ml per well of the same media. Fluorescence was acquired from the bottom of the plates using a POLARstar OPTIMA microplate reader at excitation/emission wavelengths of 485/520 nm. Background fluorescence was determined in the wells containing no hMSCs but a monolayer of endothelial cells. Total cell load was estimated as the fluorescence from non-washed wells with added hMSCs. A minimum six wells were used to estimate background fluorescence, total cell load or fluorescence from adhered cells. Percent of bound cells was calculated as a ratio between fluorescence of adhered cells and total cell load after subtraction of background fluorescence. Data from five independent preparations of hMSC spheroids and four independent preparations of hMSCs grown as a monolayer were statistically processed with the SigmaStat software package (Sigma).

### EXAMPLE 2

Expression of Cell Surface Markers by hMSCs

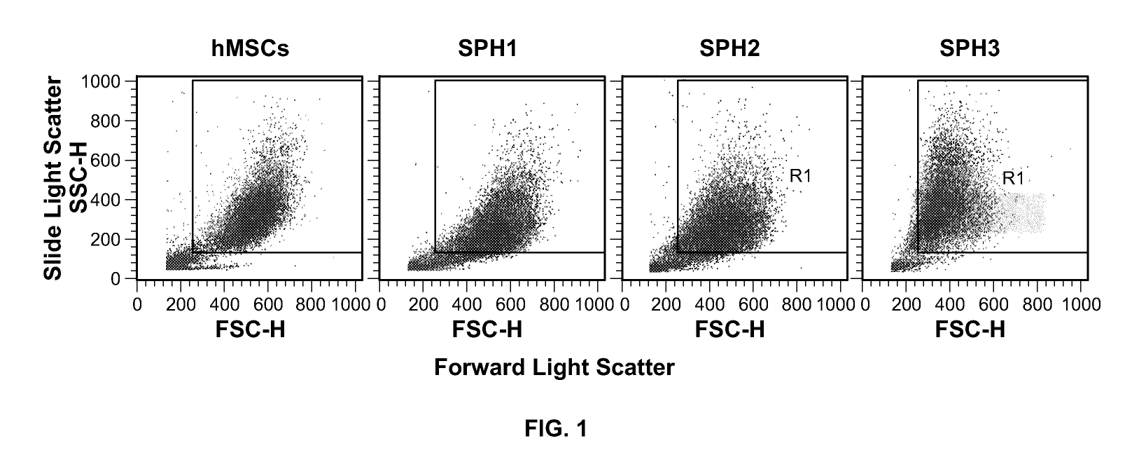

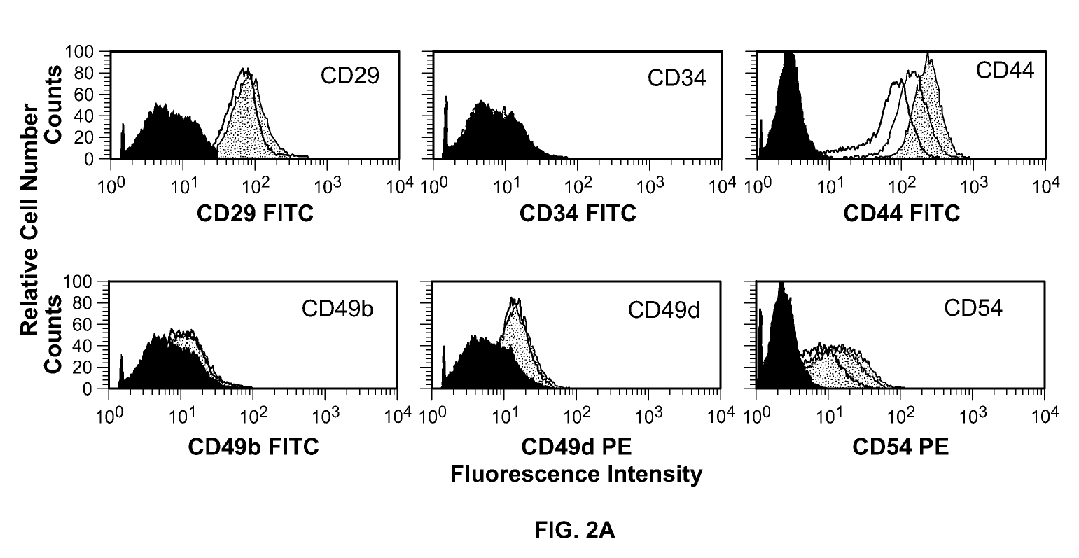

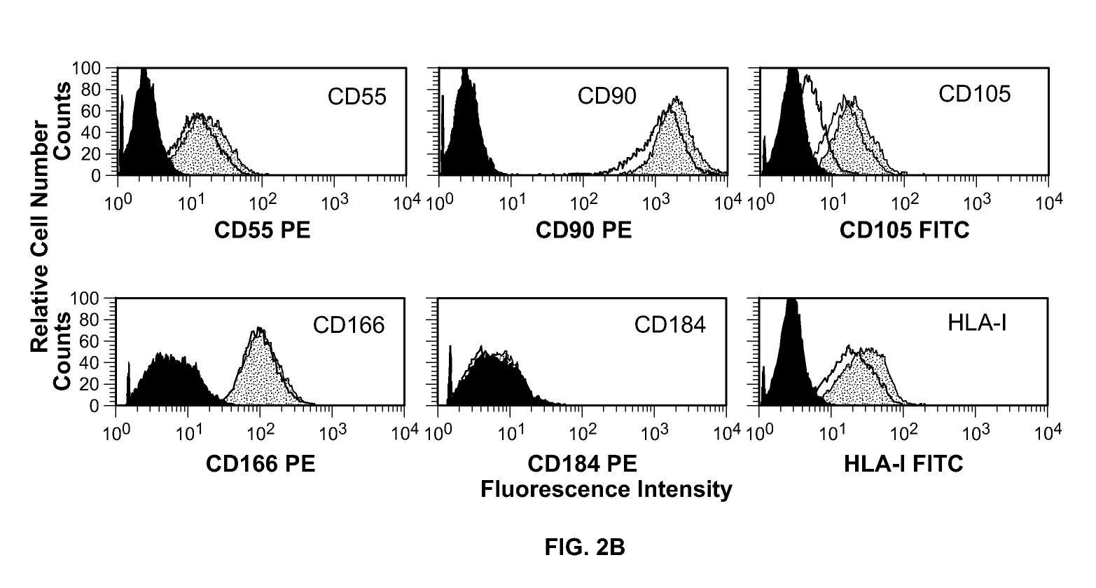

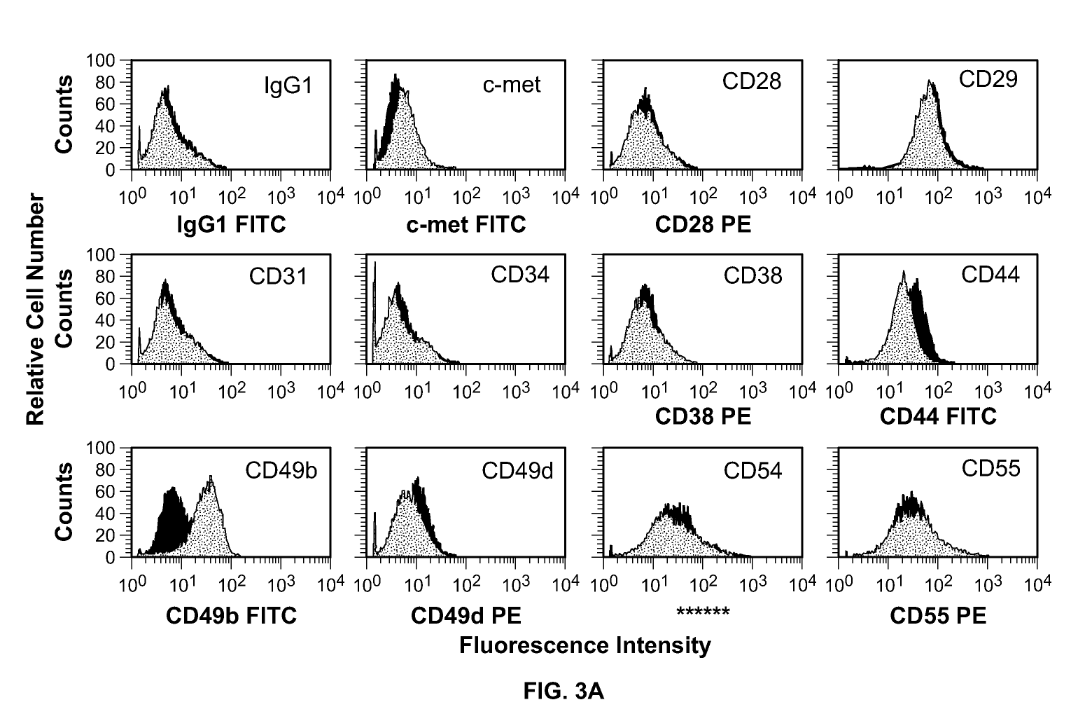

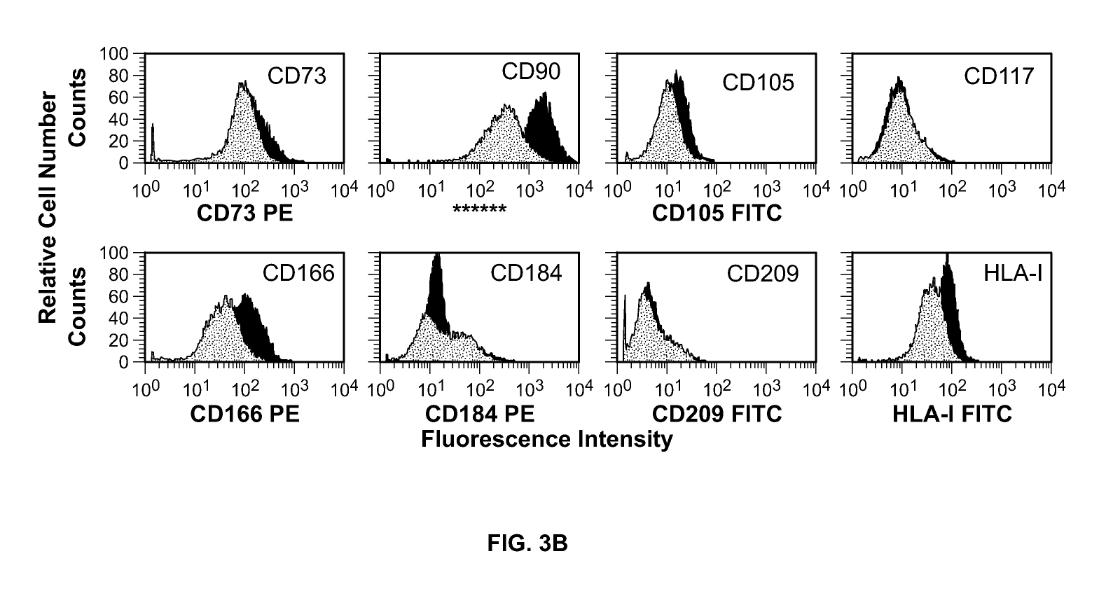

Cells dissociated from a monolayer or the spheroids gave rise to homogeneous populations. Cells from spheroids had smaller size and higher granularity (FIG. 1). Expression of 19 markers commonly used to characterize hMSCs by flow cytometry (FIG. 2) was examined. HMSCs from a monolayer and the spheroids were positive for CD29, CD44, CD54, CD55, CD73, CD90, CD105, CD166 and HLA-I. Neither cells from a monolayer nor cells from the spheroids were positive for c-met, CD28, CD31, CD34, CD38, CD117 or CD209. The effects of trypsinization on the expression of cell surface markers by hMSCs were also analyzed. Treatment with trypsin for 90 min did not affect the expression of CD49d and CD166 in hMSCs. Detection of CD29, CD44, CD54, CD55, CD90, CD105 and HLA-I was sensitive to trypsin-EDTA treatment. Nevertheless, all antigens that tested positive after 5 min of trypsinization remained positive after 90 min of incubation with trypsin (FIG. 3).

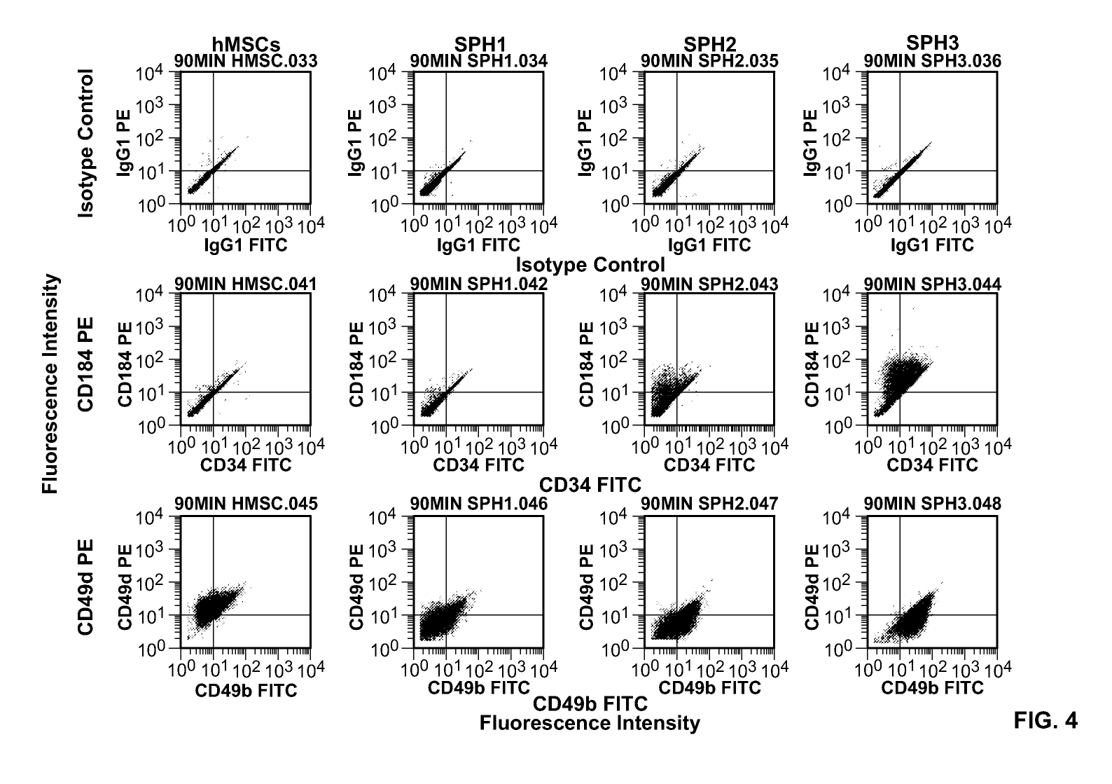

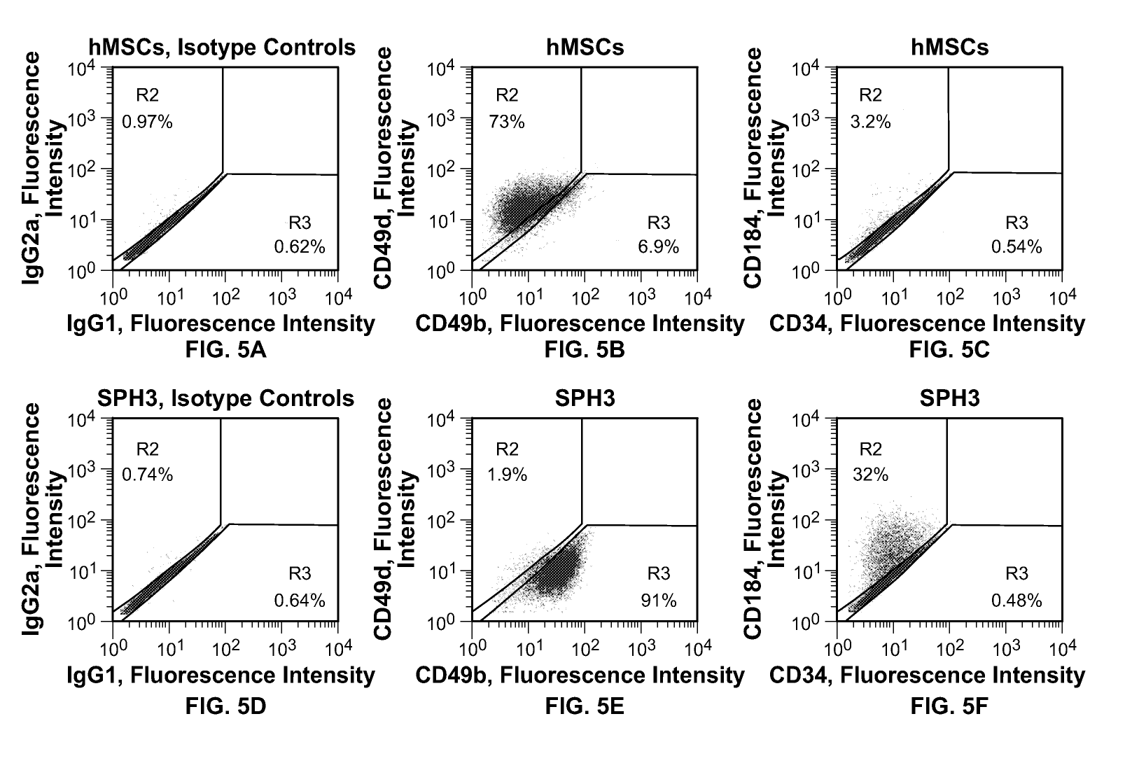

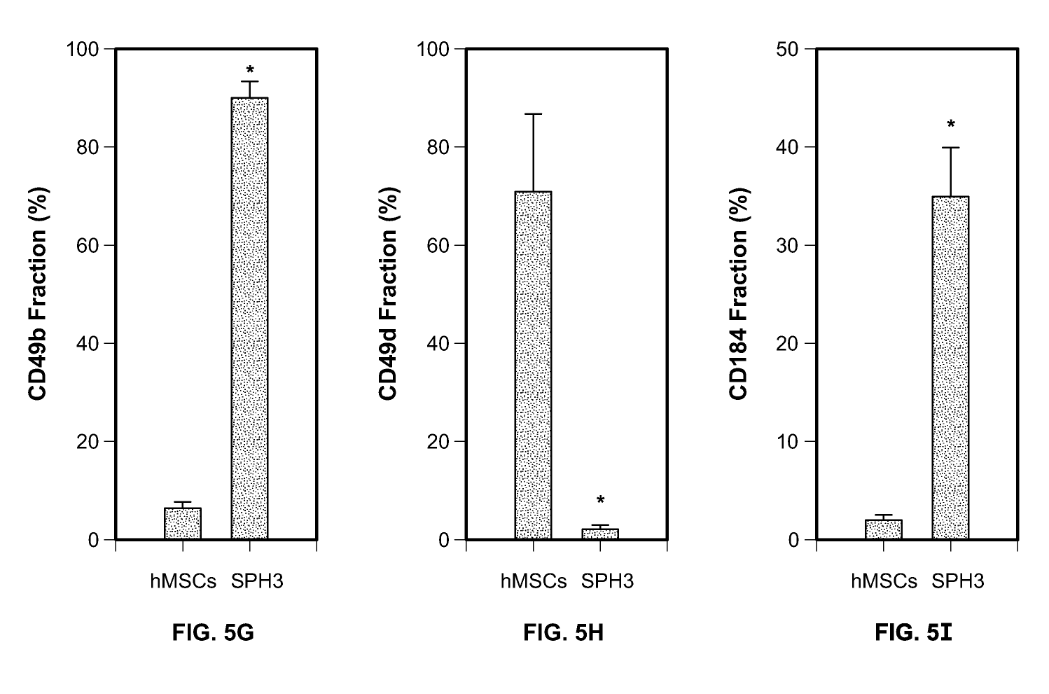

Cells from a monolayer and the spheroids showed substantial differences in the expression of CD49d (α4 integrin subunit), CD49b (α2 integrin subunit) and CD184 (CXCR4). Changes in the expression of these antigens occur gradually from day one to day three of hMSC culturing of the spheroids (FIG. 4). Representative staining of hMSCs from a monolayer and 3 day old spheroids are shown in FIG. 5A-F. Cells from the spheroids were CD49d negative and CD49b positive (FIG. 5E) while cells from a monolayer were CD49b negative and CD49d positive (FIG. 5B). HMSCs grown as a monolayer did not express CD184 (FIG. 5C). A significant fraction of cells isolated from the spheroids demonstrated positive staining for CD184 (FIG. 5F). On average 6.19±1.4% cells from a monolayer and 89.9±3.5% cells from 3 day old spheroids were positive for CD49b (FIG. 5G). Fraction of cells stained positive for CD49d was 70.77±15.9% for cells from a monolayer and 1.95±1% for cells from 3 day old hMSC spheroids (FIG. 5H). Only 2%±0.5% cells from a monolayer were CD184 positive; 35%±5% cells from 3 day old spheroids showed positive staining for CD184 (FIG. 51). The differences in the expression levels of CD49b, CD49d and CD184 were statistically significant (t-test, p-value<0.05).

Overall, flow cytometric analysis demonstrated that the pattern of antigen expression by cells from the spheroids was similar to that found for hMSCs from a monolayer. Substantial differences, however, were detected in the expression of several proteins responsible for cell adhesion and motility: CXCR4, the α2 and α4 integrin subunits.

### Example 3

Secretion of SDF-1 by hMSCs and hMSC Spheroids

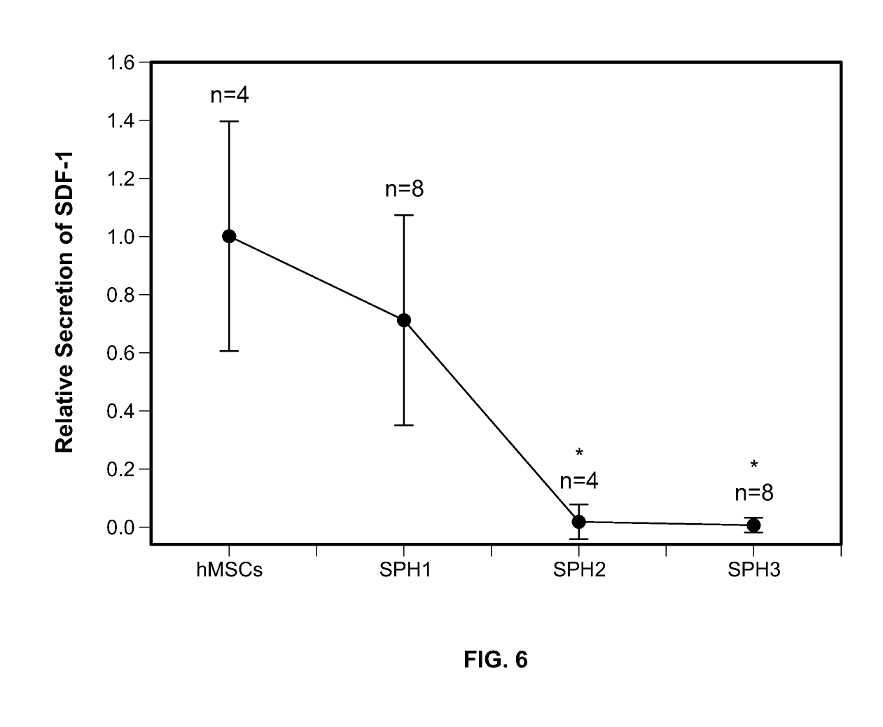

It has been reported that the expression of CXCR4 by hMSCs inversely correlates with the expression of SDF-1 (Lisignoli, G., 2006, J. Cell. Physiol. 207:364-73). The expression of SDF-1 mRNA was 10-fold down regulated in hMSC spheroids. To determine how formation of the spheroids affects SDF-1 secretion by hMSCs. SDF-1 was measured in media conditioned by a monolayer of hMSCs or 1, 2, or 3 day old hMSC spheroids. The amount of SDF-1 in conditioned media was normalized to the cell number. Relative changes in the secretion of SDF-1 are shown in FIG. 6. There was a statistically significant decline (t-test, p-value<0.05) in SDF-1 secretion by 2 and 3 day old hMSC spheroids in comparison with hMSCs from a monolayer.

### Example 4

Transfer of hMSCs from the Spheroids to a Monolayer Restores the Expression Pattern of CXCR4, the α2 and α4 integrin subunits.

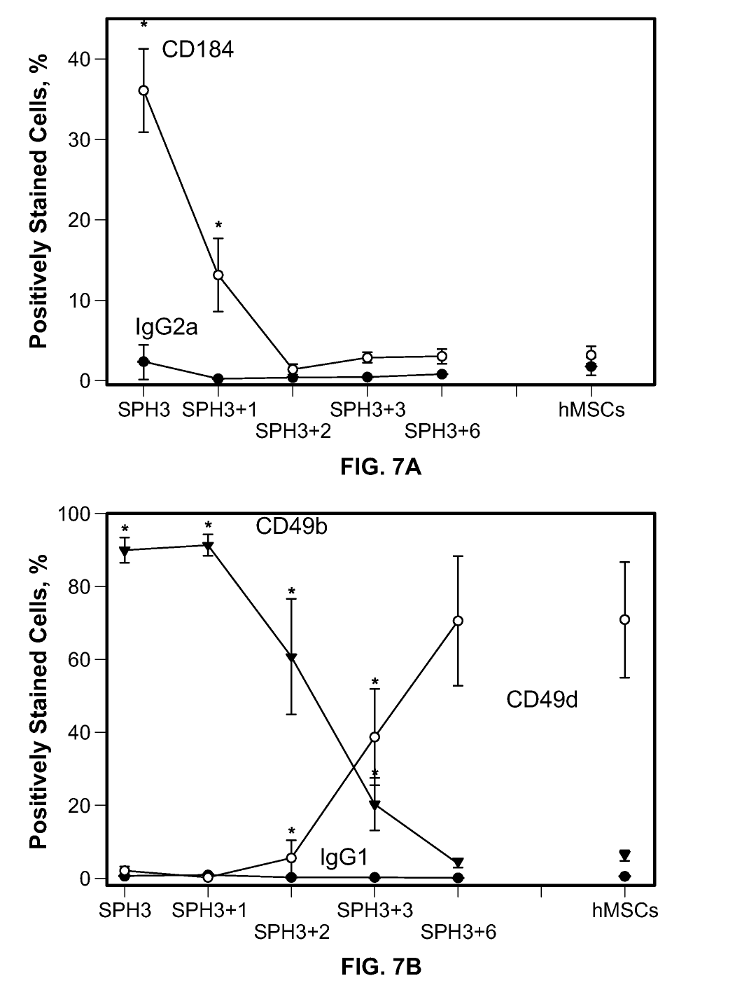

Changes in the expression of CXCR4 and the α2 and isolated from 3 day old hMSC spheroids and maintained in monolayer for 1-7 days were investigated. Expression of CXCR4 (CD184), the α2 (CD49b) and α4 (CD49d) integrin subunits were analyzed by flow cytometry. FIG. 7A shows that the expression of CXCR4 (CD184) was down regulated. Expression of the αa2 (CD49b) and α4 (CD49d) integrin subunits were also returned to the levels characteristic of a monolayer of hMSCs (FIG. 7B). Changes in the expression of CXCR4, the α2 and α4 integrin subunits are robust and occur within 48 hours.

### Example 5

SDF-1 Induces Internalization of CXCR4

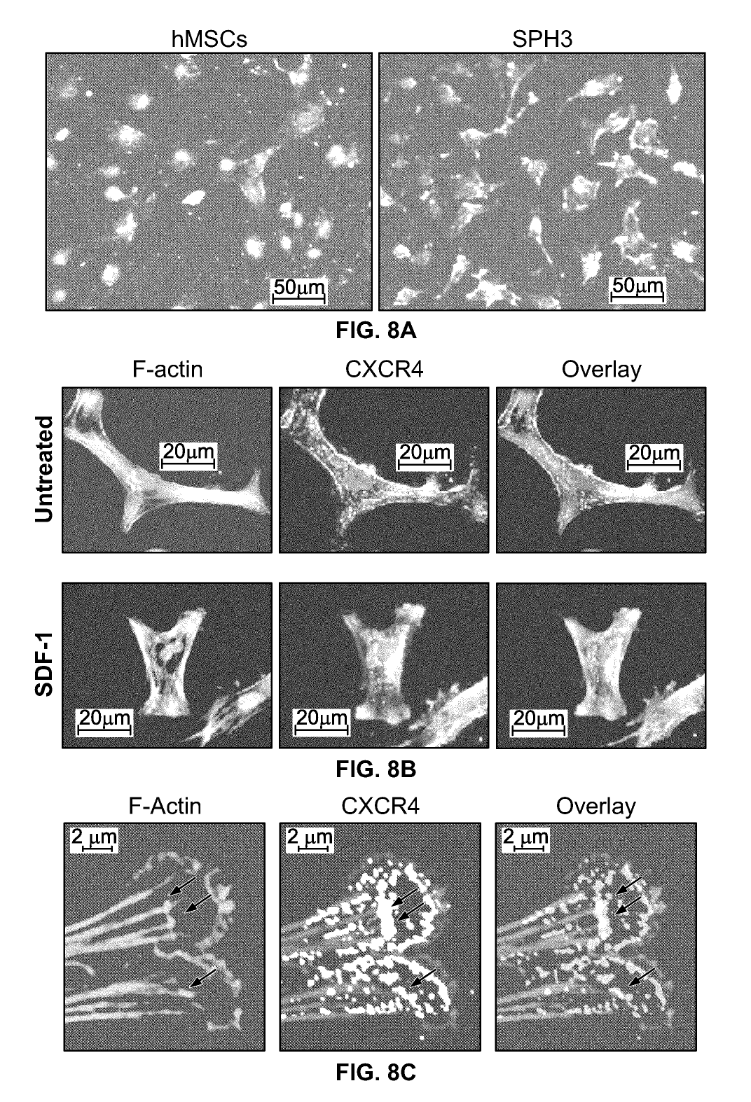

Expression of CXCR4 in a monolayer of hMSCs was too low for positive staining (FIG. 8A). Therefore the effects of SDF-1 on intracellular localization of CXCR4 were studied with cells dissociated from the spheroids. In the absence of SDF-1, CXCR4 was localized on the cell surface (FIG. 8AB). Addition of SDF-1 resulted in internalization of CXCR4 (FIG. 8B). Internalized CXCR4 was targeted to two distinct locations. The majority of the receptor was detected in the perinuclear space of hMSCs. A portion of CXCR4 was found associated with filamentous structures of hMSC lamellipodias. The greater part of CXCR4 detected in lamellipodias was not co-localized but rather positioned along the F-actin cytoskeleton (FIG. 8C). CXCR4 showed a prominent co-localization with Factin only at the tips of growing F-actin filaments, the place where F-actin contacts focal adhesion complexes (FIG. 8C).

### Example 6

SDF-1 Activates ERK-1,2 in hMSC Spheroid Cells

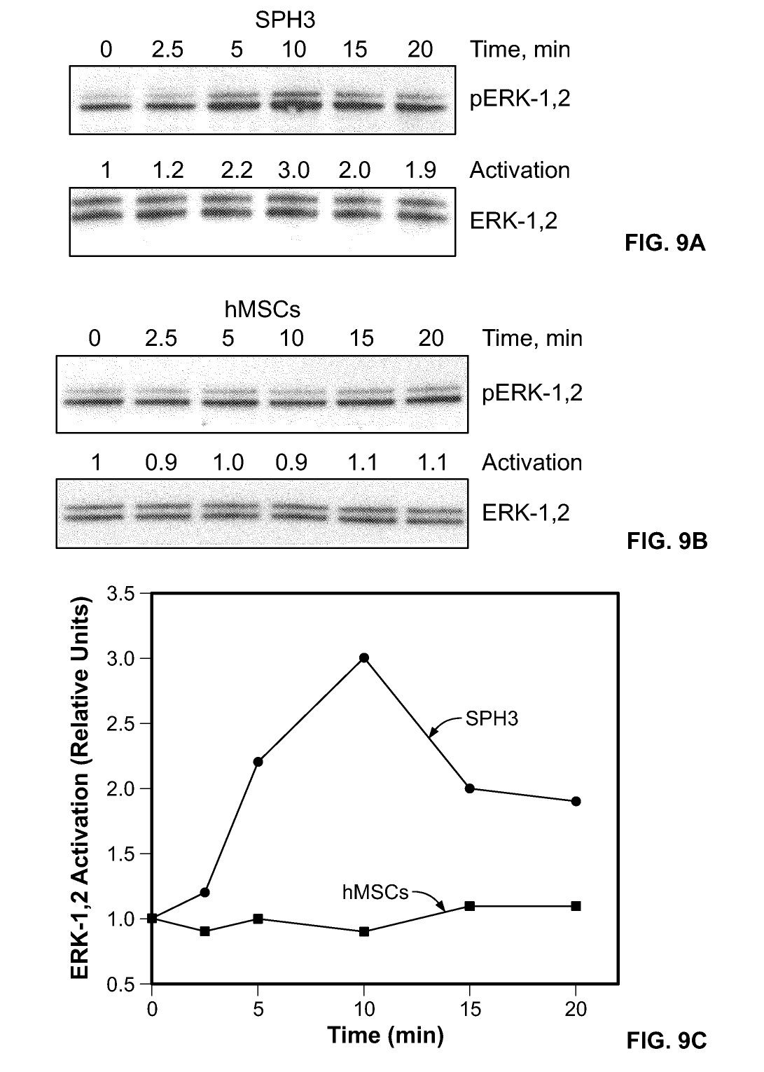

To investigate activation of signaling pathways via CXCR4 in hMSCs, we tested activation of ERK, the known downstream effector of CXCR4. Treatment of cells from a monolayer with SDF-1 does not activate ERK-1,2 (FIG. 9BC). We hypothesize that the expression level of CXCR4 in these cells is not sufficient for activation of ERK. Treatment of cells isolated from hMSC spheroids resulted in activation of ERK-1,2 suggesting that expressed CXCR4 is functionally active (FIG. 9AC).

### Example 7

CXCR4 Regulates hMSC Adhesion to Endothelial Cells

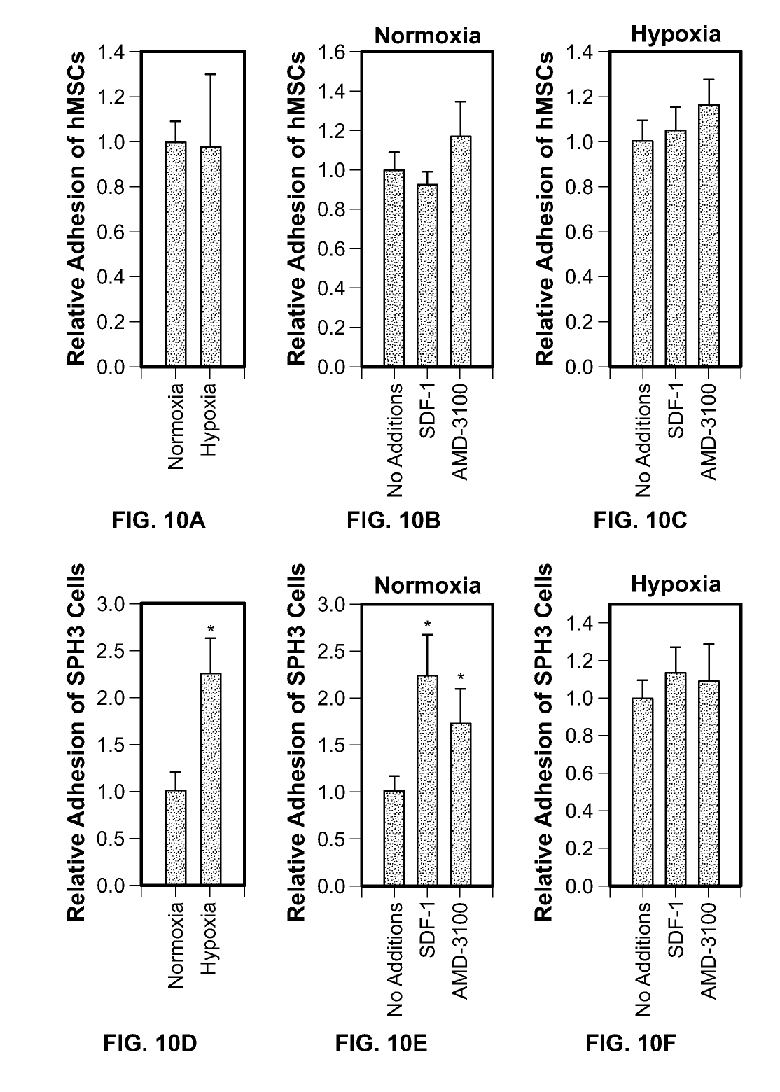

Adhesion of cells circulating in the blood stream to endothelial cells is the first and key event in cell homing. We investigated how expression of CXCR4 regulates adhesion of hMSCs to normoxic or preexposed to hypoxia endothelial cells. There was no statistically significant effect of HUVEC pre-exposure to hypoxia on the adhesion of hMSCs from a monolayer (FIG. 10A). Neither SDF-1 nor AMD3100 affected the adhesion of hMSCs from a monolayer to normoxic or pre-exposed to hypoxia endothelial cells (FIG. 10BC). In contrast, the adhesion of cells from the spheroids was stimulated 2.2±0.4 fold (t-test, p-value<0.05) by pre-exposure of HUVECs to hypoxia (FIG. 10D). SDF-1 and AMD3100 stimulated the adhesion of cells from the spheroids to normoxic endothelial cells 2.2±0.4 times and 1.7±0.4 times (ttest, p-value<0.05), respectively (FIG. 10E). Addition of SDF-1 or AMD3100 had no effect on the adhesion of cells from the spheroids to endothelial cells preexposed to hypoxia (FIG. 10F). Thus, effects of hypoxia and treatment with SDF-1 or AMD3100 on the adhesion of cells from the spheroids to HUVECs were not additive.

We claim:

1. An expanded culture of mesenchymal stem cells in which cell surface expression of CXCR4 is maintained or induced in a substantial proportion of the cells.

2. The culture of claim 1, wherein the mesenchymal stem cells are cultured in hanging drops.

3. The culture of claim 2, wherein the mesenchymal stem cells form spheroids.

4. The culture of claim 1, wherein the proportion of cells that expresses cell surface CXCR4 is at least about 20%.

5. The culture of claim 1, wherein the proportion of cells that expresses cell surface CXCR4 is at least about 30%.

6. The culture of claim 1, wherein the proportion of cells that expresses cell surface CXCR4 is at least about 35%.

7. The culture of claim 1, which comprises fetal bovine serum in an amount of about 5%.

8. The culture of claim 1, which comprises fetal bovine serum in an amount of about 5% or less.

9. The culture of claim 1, which is serum-free.

10. The culture of claim 1, wherein a substantial proportion of the cells adhere to endothelial cells exposed to hypoxia.

11. The culture of claim 3, which is dissociated from spheroids after about 2 days of hanging drop culture.

12. The culture of claim 3, which is dissociated from spheroids after about 3 days of hanging drop culture.

13. The culture of claim 3, which is dissociated from spheroids after less than about 4 days of hanging drop culture.

14. The culture of any one of claims 11 to 13, wherein the culture is promptly administered to a subject after dissociation from spheroids.

15. The culture of any one of claims 11 to 13, wherein the culture is promptly frozen after dissociation from spheroids.

16. A mesenchymal stem cell culture made by culturing mesenchymal stem cell in three dimensional culture, wherein a substantial proportion of the cell express cell surface CXCR4.

17. The mesenchymal stem cell culture of claim 16, which is started from mesenchymal stem cell monolayers.

18. The mesenchymal stem cell culture of claim 16, which is started from bone marrow mesenchymal stem cells.

19. The mesenchymal stem cell culture of claim 16, which comprises spheroids.

20. The mesenchymal stem cell culture of claim 19, which is dissociated from spheroids after about 2 days and prepared for administration to a subject.

21. The mesenchymal stem cell culture of claim 17, which is dissociated from spheroids after about 3 days and prepared for administration to a subject.

22. The mesenchymal stem cell culture of claim 17, which is dissociated from spheroids after less than about 4 days and prepared for administration to a subject.

23. A pharmaceutical composition comprising the population of mesenchymal stem cells of any one of claims 1 to 22, in which a substantial proportion of the cells express cell surface CXCR4, and a pharmaceutically acceptable carrier.

24. The pharmaceutical composition of claim 23, which is in an amount effective to treat ischemia.

25. The pharmaceutical composition of claim 23, which is in an amount effective to treat a cardiac disorder.

26. The pharmaceutical composition of claim 23, which is in an amount effective to treat a condition characterized by hypoxic tissue.

27. A method of preparing an expanded culture of mesenchymal stem cells, wherein a substantial proportion of the cells expresses cell surface CXCR4, which comprises:
a) obtaining a initial population of mesenchymal stem cells; and
b) culturing said cells under three-dimensional culture conditions for a time sufficient to induce cell surface expression of CXCR4 in a substantial proportion of the cells.

28. The method of claim 27, wherein the initial population of mesenchymal stem cells is a monolayer.

29. The method of claim 27, wherein the initial population of mesenchymal stem cells is from bone marrow.

30. The method of claim 27, wherein the cells are cultured in hanging drops.

31. The method of claim 27, wherein the three-dimensional culture conditions include fetal bovine serum at a concentration of about 5%.

32. The method of claim 27, wherein the three-dimensional culture conditions include fetal bovine serum at a concentration of less that about 5%.

33. The method of claim 27, wherein the three-dimensional culture conditions are serum-free.

34. The method of claim 27, wherein the cells are cultured for about 2 days.

35. The method of claim 27, wherein the cells are cultured for about 3 days.

36. The method of claim 27, wherein the cells are cultured for less than about 4 days.

37. The method of claim 27, wherein the proportion of cultured cells that express cell surface CXCR4 is at least about 20%.

38. The method of claim 27, wherein the proportion of cultured cells that express cell surface CXCR4 is at least about 30%.

39. The method of claim 27, wherein the proportion of cultured cells that express cell surface CXCR4 is at least about 35%.

40. The method of any one of claims 27 to 39, wherein the cells are dissociated from spheroids at the end of three dimensional culture.

41. The method of any one of claims 27 to 40, wherein the cells are promptly administered to a subject or frozen for storage at the end of three dimensional culture.

42. A method of preferentially targeting mesenchymal stem cells to hypoxic tissue in a subject comprising:
a) culturing the cells under three-dimensional culture conditions for a time sufficient to induce cell surface expression of CXCR4 in a substantial proportion of the cells; and
b) administering a therapeutically effective amount of the culture of step (a) to the subject;
whereby the cells are preferentially targeted to the hypoxic tissue.

43. The method of claim 42, wherein the subject is a human.

44. A method of preparing an expanded culture of mesenchymal stem cells that substantially express CXCR4, comprising;
obtaining mesenchymal stem cells;
culturing the mesenchymal stem cells in three dimensional culture for a sufficient time that a substantial proportion of the mesenchymal cells express cell surface CXCR4.

45. The method of claim 44, wherein the mesenchymal stem cells are obtained from a mesenchymal stem cell monolayer.

46. The method of claim 44, wherein the mesenchymal stem cells are obtained from bone marrow.

47. The method of claim 44, wherein the mesenchymal stem cells are cultured in a hanging drop for about 2 to about 3 days.

48. The method of claim 44, wherein the mesenchymal stem cells are cultured in a hanging drop for less than about 4 days.

49. A method of preparing an expanded culture of mesenchymal stem cells suitable for treatment of a cardiac disorder, comprising;
a) culturing proliferating mesenchymal stem cells in a monolayer; and
b) disrupting the monolayer and establishing the proliferating mesenchymal stem cells in three dimensional culture such that a substantial proportion of the mesenchymal cells are induced to express cell surface CXCR4;
thus providing a culture of mesenchymal stem cells suitable for treatment of a cardiac disorder.

50. The method of claim 49, wherein the mesenchymal stem cells are harvested from three-dimensional culture after about two days and dissociated for treatment of the cardiac disorder.

51. The method of claim 49, wherein the mesenchymal stem cells are harvested from three-dimensional culture after about three days and dissociated for treatment of the cardiac disorder.

52. A method of treating a subject afflicted with ischemia comprising administering to the subject a therapeutically effective amount of an expanded culture of mesenchymal stem cells, wherein a substantial proportion of the cells express cell surface CXCR4, thereby treating the ischemia.

53. A method of treating a subject afflicted with a condition characterized by hypoxic tissue comprising administering to the subject a therapeutically effective amount of an expanded culture of mesenchymal stem cells, wherein a substantial proportion of the cells express cell surface CXCR4, thereby treating the condition characterized by hypoxic tissue.

54. A method of treating a subject afflicted with a cardiac disorder comprising administering to the subject a therapeutically effective amount of an expanded culture of mesenchymal stem cells, wherein a substantial proportion of the cells express cell surface CXCR4, administering thereby treating the cardiac disorder in the subject.

55. The method of any one of claims 52 to 54, wherein the subject is a human.

56. A kit comprising mesenchymal stem cells, in which a substantial proportion of the cells express cell surface CXCR4, a physiologically acceptable carrier, and directions for administering the cells to a subject.

57. The kit of claim 56, wherein the mesenchymal stem cells are aggregated in spheroids.

58. The kit of claim 56, wherein the mesenchymal stem cells are dissociated.

59. Conditioned culture media derived from a mesenchymal stem cell culture wherein the mesenchymal stem cells are cultured in three dimensional culture.

60. The conditioned culture media of claim 59, wherein the cells are cultured in hanging drops.

61. The conditioned culture media of claim 59, wherein the three-dimensional culture conditions include fetal bovine serum at a concentration of about 5%.

62. The conditioned culture media of claim 59, wherein the three-dimensional culture conditions include fetal bovine serum at a concentration of less that about 5%.

63. The conditioned culture media of claim 59, wherein the three-dimensional culture conditions are serum-free.
